(19) United States
(12) Patent Application Publication
(10) Pub. No.: US 2026/0041331 A1
(43) Pub. Date: Feb. 12, 2026

(54) RESPIRATORY MOTION DISPLAY DEVICE, OPERATION METHOD OF RESPIRATORY MOTION DISPLAY DEVICE, AND IMAGE DIAGNOSTIC SYSTEM

(71) Applicant: FUJIFILM Corporation, Tokyo (JP)

(72) Inventors: Ayaka IKEGAWA, Tokyo (JP); Hisaaki OCHI, Tokyo (JP); Yukio KANEKO, Tokyo (JP)

(73) Assignee: FUJIFILM Corporation, Tokyo (JP)

(21) Appl. No.: 19/292,840

(22) Filed: Aug. 6, 2025

(30) Foreign Application Priority Data
Aug. 9, 2024 (JP) 2024-134614

(51) Int. Cl. A61B 5/087; A61B 5/00; A61B 5/055; A61B 6/03

(52) U.S. Cl. CPC A61B 5/0873; A61B 5/055; A61B 5/7235; A61B 6/032

(57) ABSTRACT

Provided are a respiratory motion display device, an operation method thereof, and an image diagnostic system that enable a subject to better recognize a his/her respiratory motion without moving a visual line. In a respiratory motion display device including a processor, a projector that displays a video in an aspect that is visible to a subject during an examination of the subject by an MRI apparatus, and a respiration sensor including a first camera and a second camera that detects a respiratory motion of the subject, the processor is configured to: generate a first figure that is rotationally symmetric about a fixed center and that changes in size in accordance with displacement of a respiratory waveform corresponding to the respiratory motion detected by the respiration sensor; and project the generated first figure onto a ceiling in a bore of the MRI apparatus by the projector as the video.

### CROSS-REFERENCE TO RELATED APPLICATION

The present application claims priority under 35 U.S.C § 119(a) to Japanese Patent Application No. 2024-134614 filed on Aug. 9, 2024, which is hereby expressly incorporated by reference, in its entirety, into the present application.

### BACKGROUND OF THE INVENTION

### 1. Field of the Invention

The present invention relates to a respiratory motion display device, an operation method of a respiratory motion display device, and an image diagnostic system, and particularly relates to a technique that enables a subject to better recognize a his/her respiratory motion.

### 2. Description of the Related Art

In the related art, a respiratory gating imaging method or a breath-holding imaging method is applied to an examination of abdomen of the subject using an image diagnostic apparatus such as a magnetic resonance imaging apparatus (MRI apparatus) or an X-ray computed tomography (CT) apparatus, in order to reduce motion artifacts caused by respiration of the subject.

In the respiratory gating imaging method or the like, a respiration sensor is attached to the abdomen of the subject, and data is measured only in a state in which a movement of the abdomen is small and stable (mainly exhalation). An imaging time depends on a respiratory state of the subject, and in a case where a depth of the respiration is not stable even though a respiratory cycle is stable, a deterioration of image quality is a problem.

Therefore, a technique of inducing respiration by displaying an indicator or the like to a subject in a bore of an image diagnostic apparatus has been proposed (JP2008-514371A and JP2006-158762A).

One embodiment of a method of giving an instruction to a patient, which is disclosed in JP2008-514371A, displays a first indicator that moves in a first direction and a second direction (that is, an upward direction and a downward direction) to match with respiration of the patient, and a second indicator that consists of a bar including a first line representing a minimum inhalation level of defined respiration and a second line representing a maximum inhalation level of the defined respiration. In a case where a breath-holding imaging method is performed, the patient adjusts a depth of the respiration such that the first indicator is included in the bar indicated by the second indicator, and stops the respiration.

In addition, in another embodiment of the method of giving an instruction to a patient, which is disclosed in JP2008-514371A, a curve (moving curve) representing a desired respiratory waveform to be achieved is displayed as a target, and the patient adjusts the respiration so that the first indicator is along the curve as much as possible.

An MRI apparatus described in JP2006-158762A displays a respiratory state in an aspect in which a subject is visually recognizable, and particularly displays a depth of respiration by gradations or a light-emitting location of a light spot.

### SUMMARY OF THE INVENTION

In the method disclosed in JP2008-514371A, the patient needs to adjust the depth of the respiration and the like to match with the target second indicator while looking at the first indicator that moves in the upward direction and the downward direction in accordance with the respiration of the patient. Since the first indicator moves in accordance with the respiration, there is a high possibility that a visual line moves and induces a body motion other than a respiratory motion, and there is a problem that the first indicator and the second indicator are difficult to see for the subject with poor visual acuity.

In a case of the MRI apparatus described in JP2006-158762A, there is also a problem that the visual line of the subject is moved in a case where the subject checks the gradations or the light-emitting location of the light spot which indicate the depth of the respiration, and there is a high possibility of inducing the body motion other than the respiratory motion.

The present invention has been made in view of such circumstances, and an object of the present invention is to provide a respiratory motion display device, an operation method of a respiratory motion display device, and an image diagnostic system that can further suppress the movement of the visual line of the subject and that enable the subject to better recognize a his/her respiratory motion.

An invention according to a first aspect is a respiratory motion display device comprising a processor, a display that displays a video in an aspect that is visible to a subject during an examination of the subject by an image diagnostic apparatus, and a respiration sensor that detects a respiratory motion of the subject, in which the processor is configured to: generate a first figure that is rotationally symmetric about a fixed center and that changes in size in accordance with displacement of a respiratory waveform corresponding to the respiratory motion detected by the respiration sensor; and display the generated first figure on the display as the video.

According to the first aspect of the present invention, the subject under the examination by the image diagnostic apparatus can check the respiratory motion and the depth of respiration of the subject by viewing the first figure that is rotationally symmetric about a fixed center and that changes in size in accordance with the displacement of the respiratory waveform corresponding to the respiratory motion of the subject, and especially since the center (center of symmetry) of the first figure does not move, the subject can fix the visual line in a case of viewing the first figure. As a result, it is possible to suppress the induction of the body motion other than the respiratory motion.

In a respiratory motion display device according to a second aspect of the present invention, in the first aspect, it is preferable that the processor is configured to: detect a maximum value and a minimum value of the respiratory waveform; and set the sizes of the first figure in a case where the respiratory waveform is the maximum value and the minimum value, as a maximum size and a minimum size, respectively, and generate the first figure that changes in size between the maximum size and the minimum size in accordance with the displacement of the respiratory waveform detected by the respiration sensor in a case where the respiratory waveform changes between the maximum value and the minimum value. As a result, the first figure having the maximum size and the minimum size can be displayed with an appropriate size regardless of the displacement (depth of respiration) of the respiratory waveform of each subject, and the first figure can be displayed easily to see even for a subject with weak visual acuity.

In a respiratory motion display device according to a third aspect of the present invention, in the second aspect, it is preferable that the processor is configured to match a second figure, of which an outer shape is similar to an outer shape of the first figure and which has the maximum size, with the center of the first figure to display the second figure on the display. The second figure having the maximum size is displayed as a target in a case where the respiratory waveform of the subject is at its maximum.

In a respiratory motion display device according to a fourth aspect of the present invention, in the third aspect, it is preferable that the processor is configured to make the first figure and the second figure different in at least one of a color, a line type, or brightness to display the first figure and the second figure on the display.

In a respiratory motion display device according to a fifth aspect of the present invention, in the third aspect, it is preferable that the processor is configured to match a third figure, of which an outer shape is similar to the outer shape of the first figure and which has the minimum size, with the center of the first figure to display the third figure on the display. The third figure having the minimum size is displayed as a target in a case where the respiratory waveform of the subject is at its minimum.

In a respiratory motion display device according to a sixth aspect of the present invention, in the fifth aspect, it is preferable that the processor is configured to make the first figure and the third figure different in at least one of a color, a line type, or brightness to display the first figure and the third figure on the display.

In a respiratory motion display device according to a seventh aspect of the present invention, in the fifth aspect or the sixth aspect, it is preferable that the processor is configured to: generate a respiratory waveform for respiratory gating scan based on a respiratory gating parameter set in the respiratory gating scan by the image diagnostic apparatus and on a cycle of respiration of the subject; generate a fourth figure of which an outer shape is similar to the outer shape of the first figure and which changes in size between the second figure and the third figure according to the respiratory waveform for the respiratory gating scan; and match the fourth figure with the center of the first figure to display the fourth figure on the display.

The fourth figure can induce the cycle, phase, and depth of the respiration for the subject, and the subject can perform the respiratory motion such that the first figure that changes in size according to the respiratory waveform of the subject matches with the fourth figure, and thus the subject can perform respiration suitable for the respiratory gating scan.

In a respiratory motion display device according to an eighth aspect of the present invention, in the seventh aspect, it is preferable that the processor is configured to make the first figure and the fourth figure different in at least one of a color, a line type, or brightness to display the first figure and the fourth figure on the display.

In a respiratory motion display device according to a ninth aspect of the present invention, in the seventh aspect or the eighth aspect, it is preferable that the processor is configured to display a warning on the display in a case where a deviation between the outer shapes of the first figure and the fourth figure exceeds a threshold value. In a case where the deviation between the outer shapes of the first figure and the fourth figure exceeds the threshold value, the respiratory motion is not suitable for the respiratory gating scan, and thus the warning is issued to the subject.

In a respiratory motion display device according to a tenth aspect of the present invention, in any one of the first aspect to the ninth aspect, it is preferable that the processor is configured to change a display form of the first figure in a period in which the image diagnostic apparatus images the subject. As a result, it is possible to notify the subject that the imaging period is reached.

In a respiratory motion display device according to an eleventh aspect of the present invention, in any one of the first aspect to the tenth aspect, it is preferable that the processor is configured to display a mark for fixing a visual line at the center of the first figure. As a result, it is possible to further promote the fixation of the visual line of the subject.

In a respiratory motion display device according to a twelfth aspect of the present invention, in any one of the seventh aspect to the ninth aspect, it is preferable that the processor is configured to fix the fourth figure in a period in which the subject is to hold the subject's breath, in a case where the subject holds the subject's breath and the image diagnostic apparatus images the subject.

In a respiratory motion display device according to a thirteenth aspect of the present invention, in any one of the seventh aspect to the ninth aspect, it is preferable that the first figure has a circular or regular polygonal outer shape.

An invention according to a fourteenth aspect is an image diagnostic system comprising the image diagnostic apparatus, and the respiratory motion display device of the thirteenth aspect.

In an image diagnostic system according to a fifteenth aspect of the present invention, in the fourteenth aspect, the image diagnostic apparatus includes a magnetic resonance imaging apparatus or an X-ray CT apparatus.

In an image diagnostic system according to a sixteenth aspect of the present invention, in the fourteenth aspect or the fifteenth aspect, it is preferable that the image diagnostic apparatus is an apparatus capable of performing respiratory gating scan, and the processor is configured to display at least the first figure on the display in the respiratory gating scan by the image diagnostic apparatus.

An invention according to a seventeenth aspect is an operation method of a respiratory motion display device including a processor, a display that displays a video in an aspect that is visible to a subject during an examination of the subject by an image diagnostic apparatus, and a respiration sensor that detects a respiratory motion of the subject, the operation method comprising: a step of acquiring, via the processor, a respiratory waveform corresponding to the respiratory motion detected by the respiration sensor; a step of generating, via the processor, a first figure that is rotationally symmetric about a fixed center and that changes in accordance with displacement of the acquired respiratory waveform; and a step of displaying, via the processor, the generated first figure on the display as the video.

According to the present invention, the subject under the examination by the image diagnostic apparatus can better recognize the respiratory motion of the subject without moving the visual line by viewing the first figure that is rotationally symmetric about a fixed center and that changes in size in accordance with the displacement of the respiratory waveform corresponding to the respiratory motion of the subject.

### BRIEF DESCRIPTION OF THE DRAWINGS

FIG. 1 is a perspective view showing an appearance of a magnetic resonance imaging apparatus (MRI apparatus) to which a respiratory motion display device according to an embodiment of the present invention is applied.

FIG. 2 is a view showing a schematic configuration of an inside of the MRI apparatus shown in FIG. 1.

FIG. 3 is a view showing an external configuration of a main part of the respiratory motion display device according to the embodiment of the present invention.

FIG. 4 is a block diagram showing an embodiment of an image diagnostic system according to the embodiment of the present invention.

FIG. 5 is a view showing an example of a respiratory waveform 240 showing a respiratory motion of a subject.

FIG. 6 is a view showing a part of a video (figure) projected by a projector, and is a view particularly showing an example of a second figure C2 and a third figure C3 which correspond to a maximum value and a minimum value of the respiratory waveform, respectively.

FIG. 7 is a view showing the figure projected by the projector, and is a view particularly showing a figure in which a first figure C1, the second figure C2, and the third figure C3 are combined.

FIG. 8 is a flowchart showing an embodiment of an operation method of a respiratory motion display device according to the embodiment of the present invention.

FIG. 9 is a waveform diagram showing an example of a respiratory waveform for respiratory gating scan in a case where the respiratory gating scan is performed by the MRI apparatus.

FIG. 10 is a view showing the figure projected by the projector, and is a view particularly showing a figure in which the first figure C1, the second figure C2, and a fourth figure C4 are combined.

FIG. 11 is a view showing the figure projected by the projector, and is a view particularly showing an example of a figure in a case where respiration of the subject does not follow respiration for respiration induction.

FIG. 12 is a view showing the figure projected by the projector, and is a view particularly showing another example of the figure in a case where the respiration of the subject does not follow the respiration for respiration induction.

FIG. 13 is a view showing the figure projected by the projector, and is a view particularly showing a change in a display form of a first figure depending on whether or not respiratory gating measurement is being performed.

FIG. 14 is a view showing a part of the figure projected by the projector, and is a view in which a mark for fixing a visual line is particularly displayed.

FIG. 15 is a view showing the figure projected by the projector, and is a view particularly showing a figure in which a first figure H1 and a second figure H2 are combined.

### DESCRIPTION OF THE PREFERRED EMBODIMENTS

Hereinafter, preferred embodiments of a respiratory motion display device, an operation method of a respiratory motion display device, and an image diagnostic system according to an embodiment of the present invention will be described with reference to the accompanying drawings.

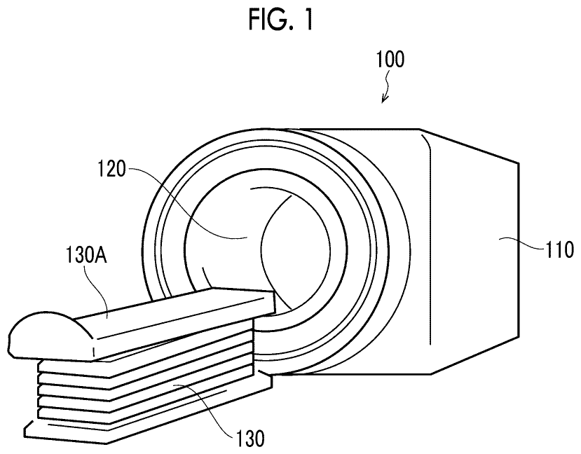

FIG. 1 is a perspective view showing an appearance of a magnetic resonance imaging apparatus (MRI apparatus) to which a respiratory motion display device according to an embodiment of the present invention is applied.

An MRI apparatus 100 shown in FIG. 1 comprises a gantry 110 and a bed 130 comprising a top plate 130A disposed on a front side of a bore 120 which is a cylinder imaging space provided in the gantry 110.

### Internal Configuration of MRI Apparatus

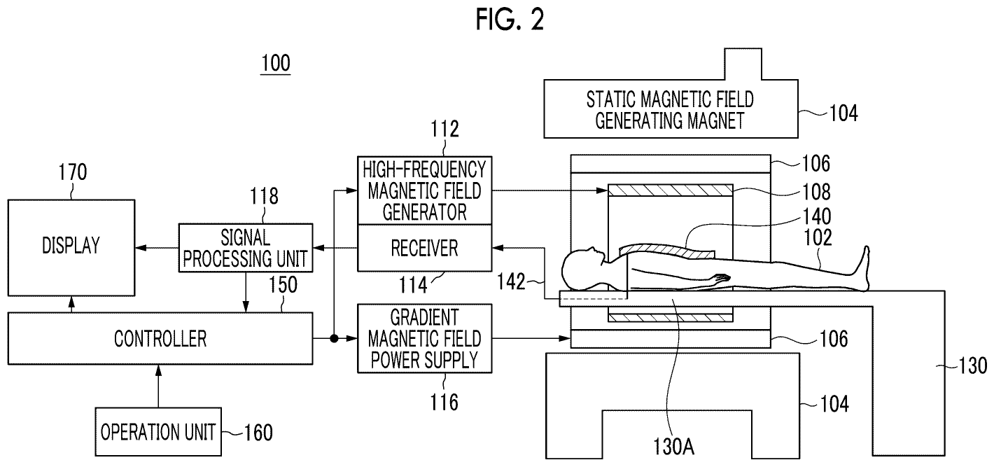

FIG. 2 is a view showing a schematic configuration of an inside of the MRI apparatus shown in FIG. 1.

As shown in FIG. 2, the MRI apparatus 100 comprises a static magnetic field generating magnet 104 that generates a uniform static magnetic field in an imaging space in which a subject 102 is disposed, a gradient coil (GC) 106, a radio frequency (RF) coil (transmission coil) 108, a receive coil 140, a high-frequency magnetic field generator 112, a receiver 114, and a gradient magnetic field power supply 116.

The gradient coil 106 includes gradient coils in three directions of X, Y, and Z directions, and generates the gradient magnetic field pulse in the imaging space in response to a signal from the gradient magnetic field power supply 116. The transmission coil 108 generates a high-frequency magnetic field that generates a nuclear magnetic resonance (NMR) signal in the atomic nuclei of atoms constituting the tissue of the subject 102 in response to a signal from the high-frequency magnetic field generator 112.

The receive coil 140 detects the NMR signal generated from the subject 102. The detected NMR signal is transmitted to the receiver 114 via a signal cable 142. The measurement data (raw data) is generated by performing analog-to-digital (AD) conversion by an AD converter in the receiver 114.

In addition, the MRI apparatus 100 further comprises a signal processing unit 118, a controller 150, an operation unit 160, and a display 170.

The signal processing unit 118 performs inverse Fourier transform on the measurement data generated by the receiver 114 to reconstruct an image, and outputs the reconstructed image signal to the controller 150 and the display 170. In FIG. 2, an example in which the receive coil 140 is connected to the signal processing unit 118 and the controller 150 via the signal cable 142 has been described, but the connection between the receive coil 140 and the signal processing unit 118 and/or the controller 150 is not limited to wired and may be wireless. As an example of the wireless connection, the receive coil 140 further includes an AD converter and a wireless communication module, and the digital data (for example, measurement data) generated by the receive coil 140 is wirelessly transmitted to the wireless communication module in the signal processing unit 118 and/or the controller 150.

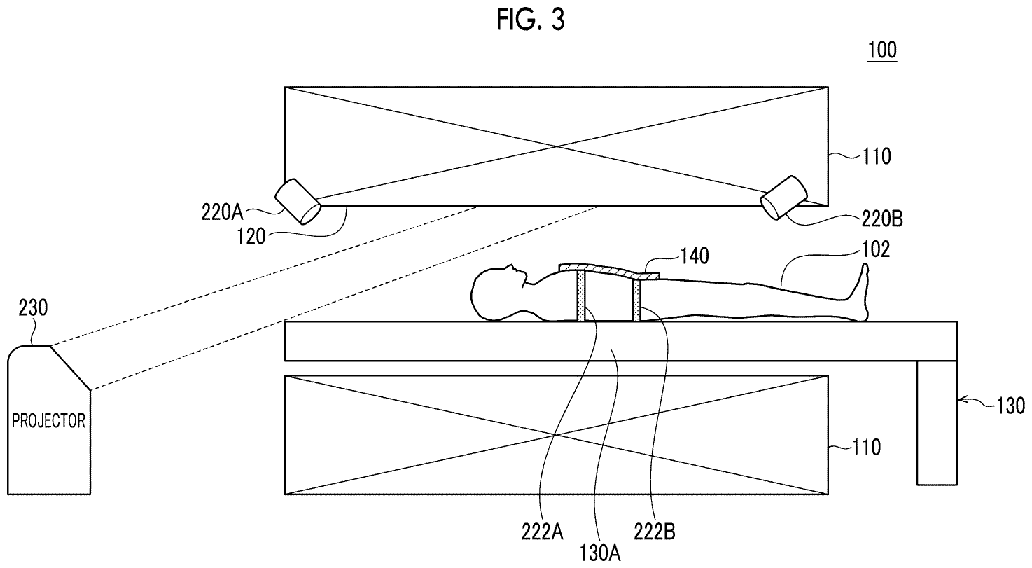

The controller 150 has a measurement controller and a calculation unit (which are not shown) and controls the entire apparatus including the high-frequency magnetic field generator 112, the gradient magnetic field power supply 116, and the display 170. The display 170 displays the reconstructed image and the video of the subject 102 captured by a first camera 220A and a second camera 220B which are shown in FIG. 3, and functions as a part of a user interface in a case where the operator inputs various parameters and the like.

The controller 150 receives the position and the size of the specific region and the protocol (imaging plan) of the examination by the operator operating the operation unit 160, sends commands to the high-frequency magnetic field generator 112 and the gradient magnetic field power supply 116 according to the pulse sequence corresponding to the imaging plan, and causes the high-frequency magnetic field and the gradient magnetic field to be generated, respectively.

In the protocol of the abdominal examination, respiratory gating measurement or breath-holding measurement is used. In a case where the respiratory gating measurement or the breath-holding measurement is performed, the respiration sensor is attached to the subject 102, and the controller 150 causes the imaging to be performed in synchronization with the signal from the respiration sensor.

In addition, the controller 150 generates a file of a format for a medical image from the image signal designated by the operation unit 160 among the image signals processed by the signal processing unit 118, and registers the file in an image database (not shown) or the like.

The signal processing unit 118 and the controller 150 can be realized by, for example, a computer comprising a processor such as a central processing unit (CPU) and a memory that stores control programs and parameters, and the like, executing calculation or control programs.

FIG. 3 is a view showing an external configuration of a main part of the respiratory motion display device according to the embodiment of the present invention. Note that in FIG. 3, parts common to those in FIGS. 1 and 2 are denoted by the same reference numerals, and detailed description thereof will be omitted.

In FIG. 3, the first camera 220A and the second camera 220B that image the subject 102 in the bore 120 are disposed in the gantry 110. The respiratory motion of the subject 102 can be detected by analyzing the videos from the first camera 220A and the second camera 220B. That is, the first camera 220A and the second camera 220B function as a part of a respiration sensor that detects a respiratory motion of the subject 102.

In addition, respiration bands 222A and 222B are respectively mounted on the chest and the abdomen of the subject 102 and function as a part of a respiration sensor that detects a shape change (respiratory motion) in the chest and the abdomen.

The respiration sensor may be a non-contact type using the first camera 220A and the second camera 220B, or a contact type using the respiration bands 222A and 222B. In addition, the number of cameras and the number of respiration bands are not limited to the embodiment shown in FIG. 3.

Further, the respiration sensor may use a sheet in which a plurality of pressure sensors to be laid under the subject are incorporated. Respiratory information of the subject can be obtained from the pressure signal detected by the pressure sensor in the sheet in response to the respiratory motion of the subject 102.

Furthermore, the respiration sensor may acquire the respiratory information of the subject 102 from an HF signal of an HF resonator by incorporating the motion sensor (HF resonator) in the top plate 130A of the bed 130.

A projector 230 projects a video onto the ceiling in the bore 120 and functions as a display that displays the video in an aspect that is visible to the subject 102 during the examination of the subject 102.

### Image Diagnostic System

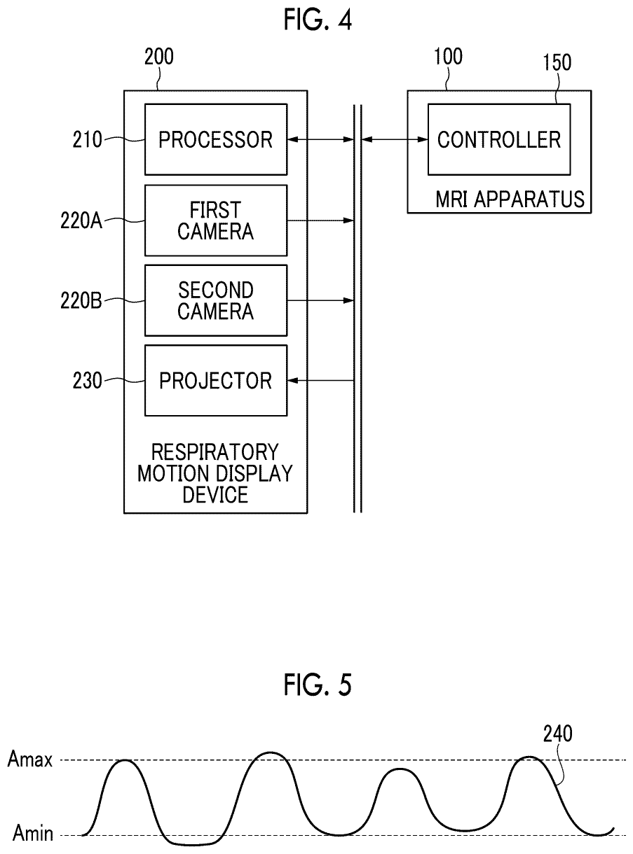

FIG. 4 is a block diagram showing an embodiment of an image diagnostic system according to the embodiment of the present invention.

The image diagnostic system shown in FIG. 4 comprises the MRI apparatus 100 which is an image diagnostic apparatus, and a respiratory motion display device 200. The configuration of the MRI apparatus 100 is specifically shown in FIGS. 1 and 2.

The respiratory motion display device 200 is configured with a processor 210, the first camera 220A and the second camera 220B, and the projector 230.

Since the first camera 220A and the second camera 220B are used as a part of the respiration sensor in the respiratory gating measurement or the breath-holding measurement, the cameras provided in the MRI apparatus 100 may be used. In addition, the respiration bands 222A and 222B may be used as the respiration sensors instead of the first camera 220A and the second camera 220B.

The processor 210 is composed of a CPU or the like, performs overall control of each unit of the respiratory motion display device 200, and executes various types of processing including processing of generating a video to be projected from the projector 230.

The processor 210 and the controller 150 of the MRI apparatus 100 can communicate with each other, and in a case where the MRI apparatus 100 performs the respiratory gating measurement or the breath-holding measurement, the processor 210 projects a video showing the respiratory motion of the subject 102 from the projector 230 onto the ceiling in the bore 120. In addition, in a case in which the MRI apparatus 100 performs imaging of a part that is not affected by the respiratory motion, such as the head, the processor 210 can project a video for relaxing the subject 102 from the projector 230 instead of the video showing the respiratory motion.

In a case where the signal processing unit 118 and the controller 150 of the MRI apparatus 100 are configured by the computer comprising the processor and the memory as described above, the processor of the MRI apparatus 100 may function as the processor 210 of the respiratory motion display device 200.

### First Embodiment of Respiratory Motion Display Device

Next, a first embodiment of the respiratory motion display device according to the embodiment of the present invention will be described.

In a case where the respiratory gating measurement or the breath-holding measurement is performed by the MRI apparatus 100, the processor 210 of the respiratory motion display device 200 shown in FIG. 4 analyzes the video of the subject 102 captured by the first camera 220A and the second camera 220B and acquires the respiratory waveform indicating the respiratory motion of the subject 102.

FIG. 5 is a view showing an example of a respiratory waveform 240 showing the respiratory motion of the subject 102.

The processor 210 detects a maximum value Amax and a minimum value Amin of the respiratory waveform 240. In this case, the average value of a plurality of maximum values and the average value of a plurality of minimum values of the respiratory waveforms 240 of a plurality of cycles can be set as the maximum value Amax and the minimum value Amin, respectively. It is preferable that the detection of the maximum value Amax and the minimum value Amin of the respiratory waveform 240 is performed before the respiratory gating measurement or the breath-holding measurement is started, but the maximum value Amax and the minimum value Amin may be updated by performing the detection even during the respiratory gating measurement or the breath-holding measurement.

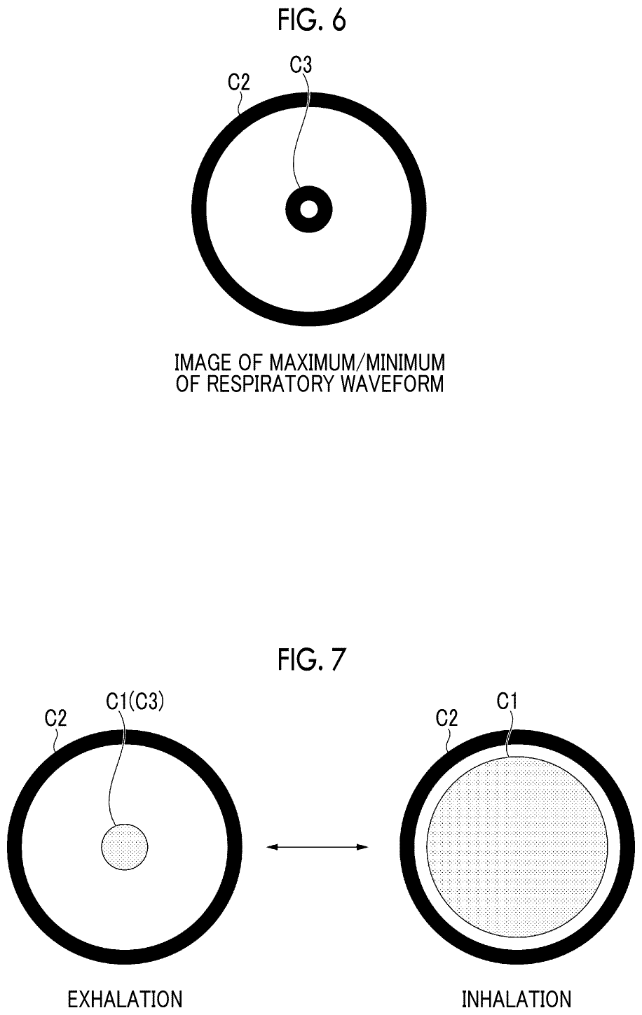

FIG. 6 is a view showing a part of a video (figure) projected by the projector 230, and is a view particularly showing an example of a second figure C2 and a third figure C3 which correspond to a maximum value and a minimum value of the respiratory waveform, respectively.

As shown in FIG. 6, the second figure C2 and the third figure C3 are two concentric circles of large and small sizes. In addition, the second figure C2 and the third figure C3 are circles of large and small that have the maximum size and the minimum size, which are fixed sizes set in advance, regardless of the displacement (depth of respiration) of the respiratory waveform for each subject.

The second figure C2 and the third figure C3 are parts of the figure projected onto the ceiling in the bore 120 from the projector 230. In a case where the size of the projection region projected onto the ceiling in the bore 120 from the projector 230 is, for example, 20 cm×20 cm, the diameter of the second figure C2 can be set to about 15 cm. As a result, even a subject with weak visual acuity who cannot wear glasses in the MRI apparatus 100 can visually recognize the second figure C2 and the third figure C3 satisfactorily.

Next, the processor 210 acquires the respiratory waveform (respiratory waveform signal) 240 corresponding to the respiratory motion of the subject 102 by analyzing the videos from the first camera 220A and the second camera 220B.

The processor 210 generates a first figure C1 that changes in size in accordance with the displacement of the respiratory waveform 240, based on the displacement (height) of the respiratory waveform 240, the maximum value Amax and the minimum value Amin (see FIG. 5) of the respiratory waveform 240, and the size of the second figure C2 and the size of the third figure C3, which are sequentially acquired.

FIG. 7 is a view showing the figure projected by the projector 230, and is a view particularly showing a figure in which the first figure C1, the second figure C2, and the third figure C3 are combined.

The first figure C1 is a figure that has an outer shape similar to an outer shape of the second figure C2 and the third figure C3, and is a figure that is combined with the second figure C2 and the third figure C3 to match with the centers of the second figure C2 and the third figure C3. That is, the first figure C1 has a concentric circular outer shape that matches with the centers of the second figure C2 and the third figure C3.

In addition, it is preferable that the first figure C1 differs from the second figure C2 and the third figure C3 in at least one of a color, a line type, or brightness. The first figure C1 shown in FIG. 7 is filled with a color and/or brightness different from those of the second figure C2 and the third figure C3.

The first figure C1 on the left side of FIG. 7 shows a case of exhalation in which the respiratory waveform is a minimum value, and the first figure C1 on the right side of FIG. 7 shows a case of inhalation in which the respiratory waveform is a maximum value.

The first figure C1 changes in size (size of the circle) in accordance with the displacement of the respiratory waveform corresponding to the respiratory motion of the subject. In a case where the displacement of the respiratory waveform is large and matches with the maximum value Amax of the respiratory waveform 240 measured in advance, the first figure C1 is enlarged to match with the outer shape of the second figure C2. On the other hand, in a case where the displacement of the respiratory waveform is small and matches with the minimum value Amin of the respiratory waveform 240 measured in advance, the first figure C1 is reduced to match with the outer shape of the third figure C3.

In FIG. 7, while the outer shape of the first figure C1 in the case of exhalation and the outer shape of the third figure C3 having the minimum size match with each other, the outer shape of the first figure C1 in the case of inhalation is slightly smaller than the second figure C2 having the maximum size.

This is because the displacement of the respiratory waveform in the case of exhalation matches with the minimum value Amin of the respiratory waveform 240, whereas the displacement of the respiratory waveform in the case of inhalation is slightly smaller than the maximum value Amax of the respiratory waveform 240.

In a case where the displacement of the respiratory waveform 240 in the exhalation is smaller than the minimum value Amin of the respiratory waveform 240, the outer shape of the first figure C1 is smaller than the outer shape of the third figure C3. In a case where the displacement of the respiratory waveform 240 in the inhalation is larger than the maximum value Amax of the respiratory waveform 240, the outer shape of the first figure C1 is larger than the outer shape of the second figure C2.

In addition, in FIG. 7, the third figure C3 having the minimum size as the target of the exhalation is not visible because it overlaps with the first figure C1. However, for example, in a case where the first figure C1 and the third figure C3 overlap with each other, it is preferable to set the transparency in a case of combining the first figure C1 so that the third figure C3 behind the first figure C1 is also visible, or to combine the third figure C3 on the first figure C1.

### Operation Method of Respiratory Motion Display Device

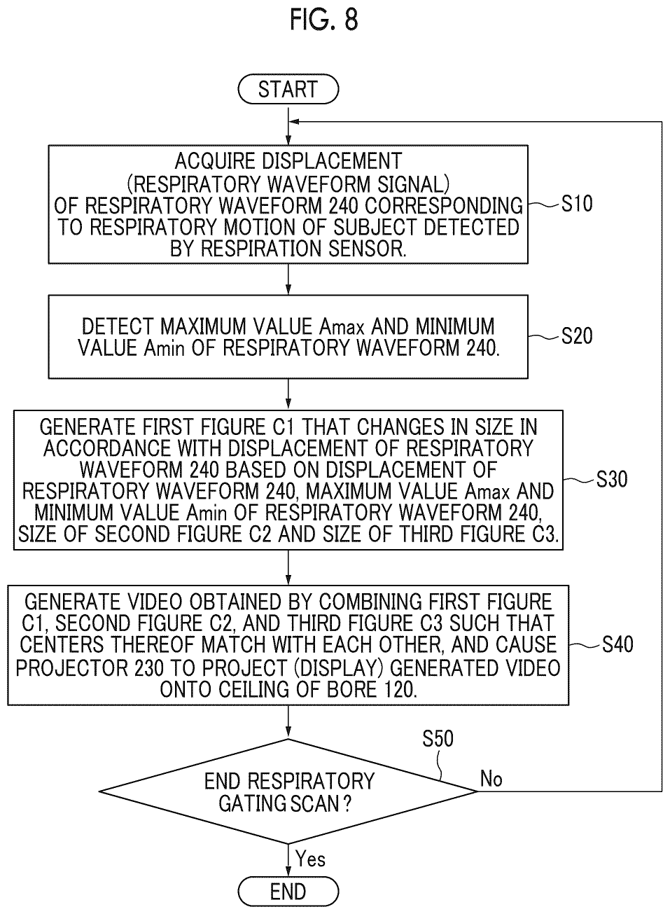

FIG. 8 is a flowchart showing an embodiment of the operation method of a respiratory motion display device according to the embodiment of the present invention, and shows processing contents and a processing procedure of the processor 210 of the respiratory motion display device 200 of the first embodiment, for example, in a case where the MRI apparatus 100 performs respiratory gating scan.

In FIG. 8, the processor 210 acquires the displacement of the respiratory waveform 240 (respiratory waveform signal) corresponding to the respiratory motion of the subject 102 by analyzing the videos from the first camera 220A and the second camera 220B which function as the respiration sensors (step S10).

The processor 210 temporarily holds the displacement of the respiratory waveform 240 acquired in step S10 for a period corresponding to a plurality of cycles of the respiratory waveform 240, and detects a representative value (for example, an average value) of a plurality of maximum values in the respiratory waveform 240 of the plurality of cycles held as the maximum value Amax of the respiratory waveform 240 of the subject 102, and similarly detects a representative value of a plurality of minimum values in the respiratory waveform 240 of the plurality of cycles as the minimum value Amin of the respiratory waveform 240 of the subject 102 (step S20, see FIG. 5). The detection of the maximum value Amax and the minimum value Amin of the respiratory waveform 240 of the subject 102 is performed before the respiratory gating scan by the MRI apparatus 100 is started, but the maximum value Amax and the minimum value Amin may be detected and updated even during the respiratory gating scan.

Next, the processor 210 generates the first figure C1 that changes in size in accordance with the displacement of the current respiratory waveform 240, based on the displacement (height) of the current respiratory waveform 240 acquired in step S10, the maximum value Amax and the minimum value Amin of the respiratory waveform 240 of the subject 102 which are detected in step S20, and the size of the second figure C2 and the size of the third figure C3 (step S30).

That is, for example, in a case where the displacement of the respiratory waveform 240 of the subject 102 changes between the maximum value Amax and the minimum value Amin which are detected in step S20, the processor 210 generates the first figure C1 that changes in size (in the present example, the diameter) between the size of the second figure C2 and the size of the third figure C3 in accordance with the displacement of the respiratory waveform 240.

Since the displacement of the respiratory waveform 240 of the subject 102 does not change exactly between the maximum value Amax and the minimum value Amin (due to an error), the sizes of the first figure C1 that are the maximum and the minimum deviate from the second figure C2 and the third figure C3 by the error.

In addition, in the present example, since the second figure C2 and the third figure C3 are circles having two known sizes of large and small, the size of the second figure C2 and the size of the third figure C3 can be, for example, diameters of the two circles of large and small, and can be acquired from the memory or the like built in the processor 210.

The processor 210 generates a video obtained by combining the first figure C1 generated in step S30, the known second figure C2 and third figure C3 such that the centers thereof match with each other, sends the generated video to the projector 230, and causes the projector 230 to project (display) the video onto the ceiling of the bore 120 (step S40).

Here, the second figure C2 and the third figure C3 are known figures, respectively, and can be vector data or a bitmap corresponding to the image projected onto the ceiling of the bore 120 in the gantry 110 of the MRI apparatus 100 by the projector 230, and can be acquired from the memory or the like built in the processor 210.

Subsequently, the processor 210 determines whether or not the respiratory gating scan by the MRI apparatus 100 ends (step S50). In a case where the processor 210 determines that the respiratory gating scan does not end (in a case of “No”), the processor 210 returns to step S10 and repeatedly executes the processing of step S10 to step S50. In a case where the processor 210 determines that the respiratory gating scan ends (in a case of “Yes”), the processor 210 ends the operation of the respiratory motion display device 200. The processor 210 can determine whether or not the respiratory gating scan ends based on communication with the controller 150 of the MRI apparatus 100.

According to the first embodiment, the subject 102 can check the status of displacement of the respiratory waveform corresponding to the respiratory motion of the subject 102 by viewing the video of the first figure C1 and the like projected onto the ceiling of the bore 120, and particularly, by viewing the first figure having the fixed center even in a case where the size of the first figure changes in accordance with the displacement of the respiratory waveform, and the like, it is possible to suppress the induction of the body motion other than the respiratory motion without the subject 102 moving the visual line. In addition, the subject 102 can suppress the variation in the depth of respiration by comparing the first figure C1 of which the center matches, the second figure C2 corresponding to the maximum value of the respiratory waveform, and the third figure C3 corresponding to the minimum value of the respiratory waveform to perform the respiratory motion.

### Second Embodiment of Respiratory Motion Display Device

Next, a second embodiment of the respiratory motion display device according to the embodiment of the present invention will be described.

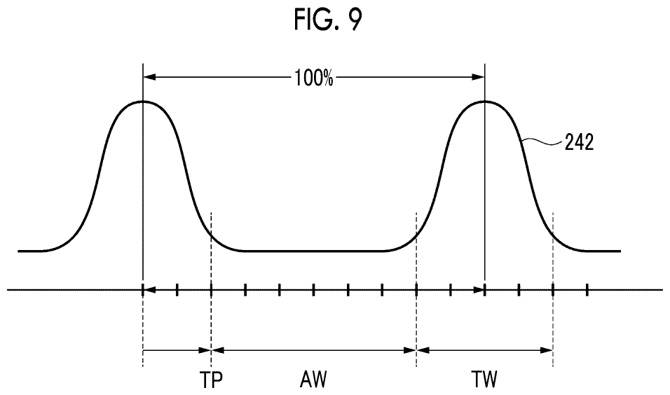

FIG. 9 is a waveform diagram showing an example of a respiratory waveform for respiratory gating scan in a case where the respiratory gating scan is performed by the MRI apparatus 100.

A respiratory waveform 242 shown in FIG. 9 is, for example, an ideal respiratory waveform suitable for respiratory gating scan, which is set in correspondence with a respiratory waveform including a cycle of respiration of the subject.

The processor 210 can generate the respiratory waveform 242 for respiratory gating scan based on the respiratory gating parameter set during the respiratory gating scan and the cycle of respiration of the subject.

The respiratory gating parameter includes, for example, a trigger point TP and a trigger window TW which are shown in FIG. 9.

In the respiratory waveform 242 shown in FIG. 9, in a case where the cycle of respiration is 100%, the trigger point TP is a point at which measurement is started from the first peak of the respiratory waveform 242, and the trigger window TW is a period from the end of the measurement to the trigger point TP of the next peak.

In the example shown in FIG. 9, the trigger point TP is a point at 20% from the first peak of the respiratory waveform 242, and the trigger window TW is a period of 40% from the end of the measurement to the trigger point TP of the next peak. In a case where the trigger window TW is 40%, an acquisition window AW other than the trigger window TW is 60%, and the measurement (imaging) is performed in the acquisition window AW.

The respiratory gating parameter may be set by the operator checking the respiratory waveform of the subject 102 and performing the setting on the operation unit 160 based on the empirical rule, or may be automatically set by the controller 150 of the MRI apparatus 100 based on the respiratory waveform of the subject 102.

In the second embodiment of the respiratory motion display device, the processor 210 generates the respiratory waveform 242 for respiratory gating scan based on the respiratory gating parameter set during the respiratory gating scan and the cycle of respiration of the subject 102.

Subsequently, the processor 210 generates a fourth figure C4 of which an outer shape is similar to the outer shape of the first figure C1 (the second figure C2 and the third figure C3) and which is for respiration induction that changes in size between the second figure and the third figure according to the respiratory waveform 242 for respiratory gating scan.

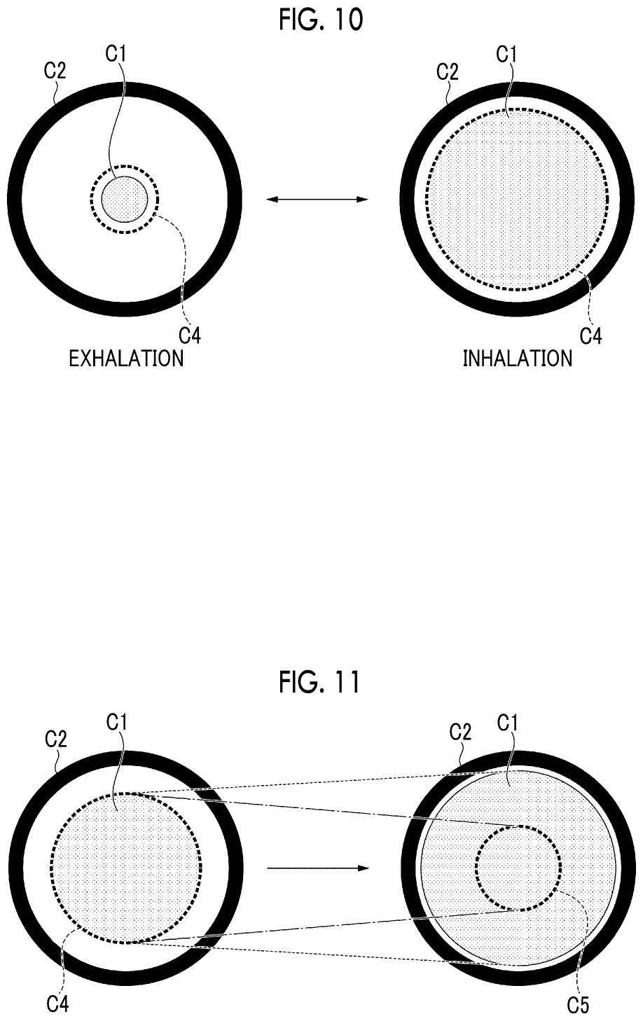

FIG. 10 is a view showing the figure projected by the projector 230, and is a view particularly showing a figure in which the first figure C1, the second figure C2, and the fourth figure C4 are combined.

The processor 210 generates the fourth figure C4 for respiration induction having a display form that is distinguishable from the first figure C1 and the like. In the example shown in FIG. 10, the fourth figure C4 is displayed by a dotted circle.

The processor 210 can make the first figure C1 and the fourth figure C4 distinguishable from each other by making the first figure C1 and the fourth figure C4 different in at least one of the color, the line type, or the brightness. In addition, in FIG. 10, the first figure C1 and the fourth figure C4 do not overlap with each other. However, regardless of the size of the first figure C1, such that the fourth figure C4 can always be visible, for example, it is preferable to set the transparency in a case of combining the first figure C1 so that the overlapped fourth figure C4 behind the first figure C1 is also visible, or to combine the fourth figure C4 on the first figure C1.

The processor 210 generates a video in which the generated fourth figure C4 for respiration induction is combined with the first figure C1 and the like to match with the center of the first figure C1 and the like, sends the generated video to the projector 230, and causes the projector 230 to project (display) the video onto the ceiling of the bore 120.

The fourth figure C4 for respiration induction changes in size between the second figure C2 and the third figure C3 according to the respiratory waveform 242 for respiratory gating scan shown in FIG. 9. That is, the fourth figure C4 has a function of changing in size according to the displacement of the respiratory waveform 242 for respiratory gating scan and inducing the cycle, phase, and depth of respiration for the subject 102.

In the state of the exhalation shown in FIG. 10, the outer shape of the first figure C1 is smaller than the outer shape of the respiratory waveform 242. As a result, the subject 102 can recognize that the phase and depth of the current respiratory waveform of the subject 102 are shifted with respect to the respiratory waveform 242 for respiratory gating scan.

In addition, in the state of the inhalation shown in FIG. 10, the outer shape of the first figure C1 matches with the outer shape of the fourth figure C4 for respiration induction, and as a result, the subject 102 can recognize that the current respiration of the subject (phase and depth of respiration) matches with the respiration for respiratory gating scan.

According to the second embodiment, since the fourth figure C4 for respiration induction that changes in size in accordance with the displacement of the respiratory waveform 242 for respiratory gating scan is displayed, the subject 102 can perform respiration suitable for respiratory gating scan by respiration so that the first figure C1 that changes in accordance with the displacement of the respiratory waveform of the subject 102 matches with the fourth figure C4 for respiration induction. As a result, in a case where the MRI apparatus 100 performs the respiratory gating scan, it is possible to reduce the motion artifact caused by the respiration of the subject 102.

In addition, in a case where the subject 102 holds his/her breath and the MRI apparatus 100 images the subject 102 (in a case where breath-holding measurement is performed), the processor 210 fixes the fourth figure C4 for respiration induction for a period in which the subject 102 should hold his/her breath.

Further, in a case where the fourth figure C4 is fixed, it is preferable to further change the display form of at least one or more figures of the first figure C1 to the fourth figure C4 and to alert the subject 102 that the period in which the respiration should be held is reached. For example, in a case where the fourth figure C4 is brightened in the data acquisition period as will be described later, it is considered that the period in which the respiration should be held is further brightened with priority over the data acquisition period to give an alert.

In a case where the breath-holding measurement is performed a plurality of times, it is preferable that the processor 210 displays the fourth figure C4 for respiration induction corresponding to the normal respiratory motion in a period between certain breath-holding measurement and the next breath-holding measurement.

### Modification Example of Second Embodiment of Respiratory Motion Display Device

FIG. 11 is a view showing the figure projected by the projector 230, and is a view particularly showing an example of a figure in a case where respiration of the subject 102 does not follow respiration for respiration induction.

FIG. 11 shows a case where the fourth figure C4 as a target of the respiration of the subject 102 during the respiratory gating scan is small (changing from inhalation to exhalation), while the first figure C1 is large (the subject 102 is further inhaling). That is, the respiratory phase of the respiratory waveform of the subject 102 is significantly shifted from the respiratory waveform 242 for respiratory gating scan.

In a case where the deviation between the outer shapes of the first figure C1 and the fourth figure C4 and the shift of the phase exceed the threshold value, the processor 210 causes the projector 230 to display a warning onto the ceiling of the bore 120. In the example shown in FIG. 11, a warning is issued by displaying a fifth figure C5 in which the display color (for example, achromatic color) of the fourth figure C4 is changed to red.

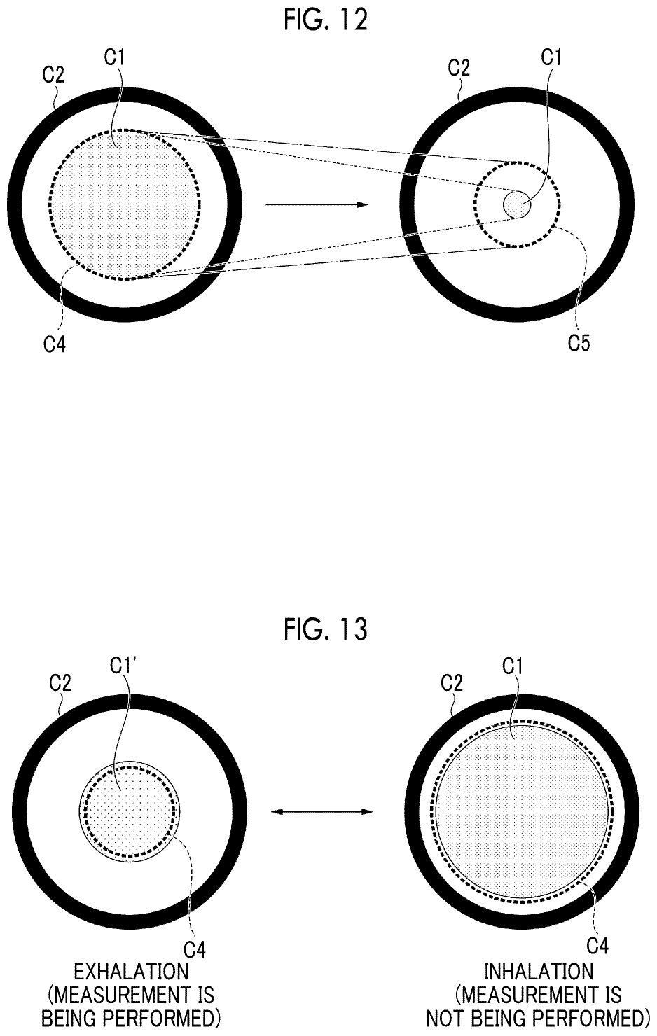

FIG. 12 is a view showing the figure projected by the projector 230, and is a view particularly showing another example of the figure in a case where the respiration of the subject 102 does not follow the respiration for respiration induction.

FIG. 12 shows a case where the fourth figure C4 as a target of the respiration of the subject during the respiratory gating scan is small and the first figure C1 is also small in the same manner, but the first figure C1 is smaller than the fourth figure C4 by more than the threshold value. That is, the displacement of the respiratory waveform of the subject 102 is lower than the target minimum value of the respiratory waveform, and as a result, the outer shape of the first figure C1 is smaller than the third figure C3 having the minimum size shown in FIG. 6.

In a case where the outer shape of the first figure C1 is smaller than the threshold value (target minimum value in the example shown in FIG. 12), the processor 210 causes the projector 230 to display the warning onto the ceiling of the bore 120. In the example shown in FIG. 12, the warning is issued by displaying the fifth figure C5 in which the display color (for example, achromatic color) of the fourth figure C4 is changed to red.

According to the modification example of the second embodiment, in a case where the respiration (respiratory waveform) of the subject 102 does not follow the respiration (respiratory waveform 242) for respiration induction beyond the threshold value, a warning is issued. Therefore, the subject 102 can be prompted to match the respiration of the subject 102 with the respiration for respiration induction.

The display of the warning is not limited to the case of changing the display form of the fourth figure C4, and may be performed by displaying a character or a warning mark, and the like.

### Third Embodiment of Respiratory Motion Display Device

FIG. 13 is a view showing the figure projected by the projector 230, and is a view particularly showing a case where a display form of a first figure is changed depending on whether or not the respiratory gating measurement is being performed.

In a case where the MRI apparatus 100 performs the respiratory gating scan, the processor 210 of the third embodiment of the respiratory motion display device changes the first figure C1 to a first figure C1′ in a period in which the MRI apparatus 100 images the subject 102. The processor 210 can change the first figure C1 to the first figure C1′ by making at least one of the color or the brightness of the first figure C1 different, for example.

As shown in FIG. 9, in a period of the trigger window TW in which the respiratory motion is performed, the first figure C1 is displayed, and in a period of the acquisition window AW in which the data measurement is performed, the first figure C1′ is displayed.

In the third embodiment, the first figure C1 is changed to the first figure C1′ as shown in FIG. 13 in the period in which the MRI apparatus 100 images the subject 102. However, the present invention is not limited thereto, and the display form may be changed as follows.

(1) The display form of the fourth figure C4 for respiration induction is changed (for example, at least one of the color or the brightness of the fourth figure C4 is changed).

(2) The first figure C1 is changed to the first figure C1′, and the display form of the fourth figure C4 is also changed.

(3) The brightness of the entire figure to be displayed is changed. For example, the data acquisition period is displayed brighter than the data non-acquisition period to alert the subject 102. Since the brightness of the display is changed, extra characters are not included, and thus the movement of the visual line of the subject 102 can be suppressed.

(4) The brightness is gradually changed (faded in) from a few seconds before the data acquisition period to the brightness of the data acquisition period. As a result, it is possible to alert the subject 102 to start of the data acquisition.

Further, in a case of an example of an embodiment in which a monitor is provided in the bore 120 instead of the projector 230, the data acquisition period can be notified to the subject 102 by changing the display brightness of the monitor.

In the example shown in FIG. 13, the first figure C1 is changed to the first figure C1′ from the end of exhalation. However, in the case of the up-trigger, the first figure C1 is changed to the first figure C1′ from the end of inhalation, and a display form opposite to the display form shown in FIG. 13 is obtained.

According to the third embodiment, it is possible to notify the subject 102 that the imaging period is reached, and the subject 102 can suppress the respiratory motion during the imaging period.

### Fourth Embodiment of Respiratory Motion Display Device

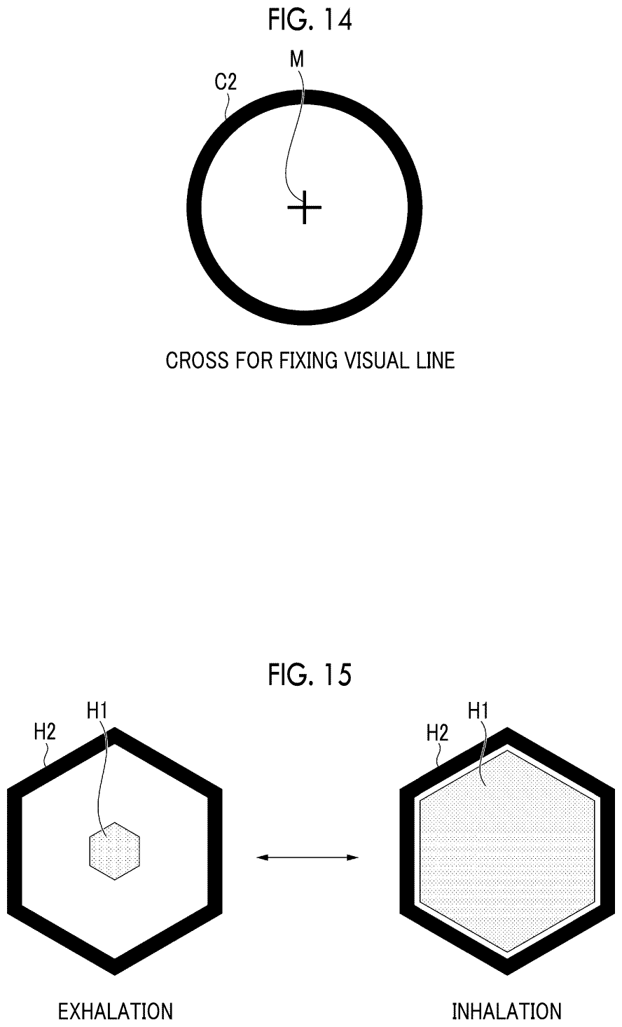

FIG. 14 is a view showing a part of the figure projected by the projector 230, and shows a case in which a mark for fixing a visual line is particularly displayed.

The processor 210 of the fourth embodiment of the respiratory motion display device displays a mark M for fixing the visual line at the center of the first figure C1 (the center of the second figure C2) shown in FIG. 7 and the like. The mark M of the present example is a cross mark, but the present invention is not limited thereto.

The first figure C1 shown in FIG. 7 and the like is also displayed, but the first figure C1 is omitted in FIG. 14. In addition, it is preferable that the mark M is combined on the first figure C1 so that the mark M is always visible even in a case where the first figure C1 is displayed.

According to the fourth embodiment, it is possible to further promote the fixation of the visual line of the subject 102 in a case of visually recognizing the first figure C1 or the like, and it is possible to prevent the visual line movement that induces the body motion including the movement of the head.

### OTHERS

The figure including the first figure C1 of the present embodiment has a circular outer shape, but is not limited thereto, and can be a figure that is rotationally symmetric about a fixed center, for example, a regular polygon.

FIG. 15 is a view showing the figure projected by the projector 230, and is a view particularly showing a figure in which a first figure H1 and a second figure H2 are combined.

The outer shape of the first figure H1 shown in FIG. 15 is a regular hexagon, and similarly, the second figure H2 is also a regular hexagon, and the centers of the first figure H1 and the second figure H2 match with each other.

The first figure H1 corresponds to the first figure C1 shown in FIG. 7, the first figure H1 on the left side of FIG. 15 shows a case of exhalation in which the respiratory waveform is a minimum value, and the first figure H1 on the right side of FIG. 15 shows a case of inhalation in which the respiratory waveform is a maximum value.

The first figure H1 changes in size (size of the regular hexagon) in accordance with the displacement of the respiratory waveform corresponding to the respiratory motion of the subject. In a case where the displacement of the respiratory waveform is large and matches with the maximum value Amax of the respiratory waveform measured in advance, the first figure H1 is enlarged to match with the outer shape of the second figure H2. On the other hand, in a case where the displacement of the respiratory waveform is small and matches with the minimum value Amin of the respiratory waveform measured in advance, the first figure H1 is reduced to the size shown on the left side of FIG. 15.

In addition, in the present embodiment, the display that displays the first figure and the like is the projector 230 that projects the video onto the ceiling of the bore 120 in the gantry 110 of the MRI apparatus 100, but the present invention is not limited to this, and for example, a monitor such as a head-up display, a head-mounted display, a liquid crystal display in the bore 120, or an organic EL display, a monitor other than the bore 120, and a set of a mirror for viewing the monitor can be considered.

Further, the image diagnostic apparatus to which the respiratory motion display device is applied is not limited to the MRI apparatus, and may be, for example, an X-ray CT apparatus.

Furthermore, in the present embodiment, each processing is executed by any computer. In addition, any computer may execute these pieces of processing by a processor, a program, or a combination thereof. Any computer may be a general-purpose computer, a computer for a specific use, a system such as a workstation, or other hardware elements capable of executing a program.

The processor may be configured by one or a plurality of pieces of hardware, and the type of hardware is not limited. For example, the processor may be configured with hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), neural processing unit (NPU), or the like. In addition, the processor has each unit or each means that executes various types of processing in the present embodiment. In addition, the types of hardware may be a combination of different types of hardware. In a case where a plurality of pieces of hardware are configured to execute one or a plurality of pieces of processing of a certain processor, the plurality of pieces of hardware may be present in devices physically separated from each other, or may be present in the same device. In addition, in any of the embodiments, the order of each processing by the processor is not limited to the above order and may be changed as appropriate. The hardware is configured by an electric circuit (circuitry) in which circuit elements such as semiconductor elements are combined, and the like.

Further, the present embodiment may be realized by hardware, software, firmware, microcode, or a combination thereof. The software, the firmware, and the microcode are configured by a program. In addition, the program may be, for example, a program module group, and each function thereof may be realized by a processor configured to execute each function. The program may be a program code or a plurality of code segments stored in one or a plurality of non-transitory computer-readable media (for example, a storage medium or other storage). The program may be divided and stored in a plurality of non-transitory computer-readable media existing in devices physically separated from each other. The program code or the code segment may represent any combination of a procedure, a function, a subprogram, a routine, a subroutine, a module, a software package, a class, or an instruction, a data structure, or a program statement. The program code or the code segment may be connected to another code segment or a hardware circuit by transmitting and receiving information, data, an argument, a parameter, or a content of a memory.

Further, the present invention is not limited to the above-described embodiment, and it goes without saying that various modifications can be made without departing from the gist of the present invention.

### EXPLANATION OF REFERENCES

100: MRI apparatus
102: subject
104: static magnetic field generating magnet
106: gradient coil
108: transmission coil
110: gantry
112: high-frequency magnetic field generator
114: receiver
116: gradient magnetic field power supply
118: signal processing unit
120: bore
130: bed
130A: top plate
140: receive coil
142: signal cable
150: controller
160: operation unit
170: display
200: respiratory motion display device
210: processor
220A: first camera
220B: second camera
222A, 222B: respiration band
230: projector
240, 242: respiratory waveform
AW: acquisition window
Amax: maximum value
Amin: minimum value
C1, C1′, H1: first figure
C2, H2: second figure
C3: third figure
C4: fourth figure
C5: fifth figure
M: mark
S10 to S50: step
TP: trigger point
TW: trigger window

What is claimed is:

1. A respiratory motion display device comprising:
a processor;
a display that displays a video in an aspect that is visible to a subject during an examination of the subject by an image diagnostic apparatus; and
a respiration sensor that detects a respiratory motion of the subject,
wherein the processor is configured to:
generate a first figure that is rotationally symmetric about a fixed center and that changes in size in accordance with displacement of a respiratory waveform corresponding to the respiratory motion detected by the respiration sensor; and
display the generated first figure on the display as the video.

2. The respiratory motion display device according to claim 1,
wherein the processor is configured to:
detect a maximum value and a minimum value of the respiratory waveform; and
set the sizes of the first figure in a case where the respiratory waveform is the maximum value and the minimum value, as a maximum size and a minimum size, respectively, and generate the first figure that changes in size between the maximum size and the minimum size in accordance with the displacement of the respiratory waveform detected by the respiration sensor in a case where the respiratory waveform changes between the maximum value and the minimum value.

3. The respiratory motion display device according to claim 2,
wherein the processor is configured to match a second figure, of which an outer shape is similar to an outer shape of the first figure and which has the maximum size, with the center of the first figure to display the second figure on the display.

4. The respiratory motion display device according to claim 3,
wherein the processor is configured to make the first figure and the second figure different in at least one of a color, a line type, or brightness to display the first figure and the second figure on the display.

5. The respiratory motion display device according to claim 3,
wherein the processor is configured to match a third figure, of which an outer shape is similar to the outer shape of the first figure and which has the minimum size, with the center of the first figure to display the third figure on the display.

6. The respiratory motion display device according to claim 5,
wherein the processor is configured to make the first figure and the third figure different in at least one of a color, a line type, or brightness to display the first figure and the third figure on the display.

7. The respiratory motion display device according to claim 5,
wherein the processor is configured to:
generate a respiratory waveform for respiratory gating scan based on a respiratory gating parameter set in the respiratory gating scan by the image diagnostic apparatus and on a cycle of respiration of the subject;
generate a fourth figure of which an outer shape is similar to the outer shape of the first figure and which changes in size between the second figure and the third figure according to the respiratory waveform for the respiratory gating scan; and
match the fourth figure with the center of the first figure to display the fourth figure on the display.

8. The respiratory motion display device according to claim 7,
wherein the processor is configured to make the first figure and the fourth figure different in at least one of a color, a line type, or brightness to display the first figure and the fourth figure on the display.

9. The respiratory motion display device according to claim 8,
wherein the processor is configured to display a warning on the display in a case where a deviation between the outer shapes of the first figure and the fourth figure exceeds a threshold value.

10. The respiratory motion display device according to claim 1,
wherein the processor is configured to change a display form of the first figure in a period in which the image diagnostic apparatus images the subject.

11. The respiratory motion display device according to claim 1,
wherein the processor is configured to display a mark for fixing a visual line at the center of the first figure.

12. The respiratory motion display device according to claim 7,
wherein the processor is configured to fix the fourth figure in a period in which the subject is to hold the subject's breath, in a case where the subject holds the subject's breath and the image diagnostic apparatus images the subject.

13. The respiratory motion display device according to claim 1,
wherein the first figure has a circular or regular polygonal outer shape.

14. An image diagnostic system comprising:
an image diagnostic apparatus; and
the respiratory motion display device according to claim 13.

15. The image diagnostic system according to claim 14,
wherein the image diagnostic apparatus includes a magnetic resonance imaging apparatus or an X-ray CT apparatus.

16. The image diagnostic system according to claim 14,
wherein the image diagnostic apparatus is an apparatus capable of performing respiratory gating scan, and
the processor is configured to display at least the first figure on the display in the respiratory gating scan by the image diagnostic apparatus.

17. An operation method of a respiratory motion display device including a processor, a display that displays a video in an aspect that is visible to a subject during an examination of the subject by an image diagnostic apparatus, and a respiration sensor that detects a respiratory motion of the subject, the operation method comprising:
a step of acquiring, via the processor, a respiratory waveform corresponding to the respiratory motion detected by the respiration sensor,
a step of generating, via the processor, a first figure that is rotationally symmetric about a fixed center and that changes in size in accordance with displacement of the acquired respiratory waveform; and
a step of displaying, via the processor, the generated first figure on the display as the video.
